(19) United States
(12) Patent Application Publication
(10) Pub. No.: US 2018/0344591 A1
(43) Pub. Date: Dec. 6, 2018

(54) EMULSION COSMETIC COMPOSITION COMPRISING OPTICAL INTERFERENCE PIGMENT AND METHOD FOR PREPARING SAME

(71) Applicant: AMOREPACIFIC CORPORATION, Seoul (KR)

(72) Inventors: Youngsun KIM, Yongin-si (KR); Jin NAM, Yongin-si (KR); Bong Soo PI, Yongin-si (KR); Yu Jin JIN, Yongin-si (KR); Soon Ae AN, Yongin-si (KR); Byungyoung KANG, Yongin-si (KR)

(21) Appl. No.: 15/988,530

(22) Filed: May 24, 2018

Related U.S. Application Data
(63) Continuation-in-part of application No. PCT/KR2016/013575, filed on Nov. 24, 2016.

(30) Foreign Application Priority Data
Nov. 27, 2015 (KR) 10-2015-0167389

(51) Int. Cl. A61K 8/06; A61K 8/29; A61K 8/20; A61K 8/81; A61K 8/58; A61Q 1/02

(52) U.S. Cl. CPC A61K 8/062; A61K 8/29; A61K 8/20; A61K 8/8117; A61K 2800/654; A61K 8/585; A61Q 1/02; A61K 2800/412; A61K 2800/436; A61K 8/8152

(57) ABSTRACT

The present description relates to an emulsion cosmetic composition comprising amphiphilic anisotropic powder and an optical interference pigment having an average particle diameter of 1-20 §-. The amphiphilic anisotropic powder comprises first polymer spheroids, which are hydrophilic, and second polymer spheroids which are hydrophobic. The first polymer spheroids and second polymer spheroids bond in a structure in which the first and second polymer spheroids penetrate each other at least partly. The first polymer spheroids have a core-shell structure, and the shell comprises a functional group.

### CROSS-REFERENCE TO RELATED APPLICATION

This application is a continuation-in part of PCT/KR2016/013575, filed Nov. 24, 2016, which claims the benefit of KR 10-2015-0167389, both of which are incorporated by reference herein in their entirety.

### TECHNICAL FIELD

The present disclosure relates to an emulsion type cosmetic composition including a light interference pigment and a method for preparing the same.

### BACKGROUND ART

Light interference pigments used for cosmetics have a plate-like or needle-like shape, unlike amorphous pigments for use in realizing general color tones to provide a visual effect imparting a pearl-like effect. In addition, it is required for such light interference pigments to be incorporated at least in a predetermined amount to impart a visual effect. When powder, such as fine interference pearl, is incorporated to an emulsion type cosmetic compound, the light interference pigment attacks the emulsion interface due to its characteristics in shape and high content, thereby making it difficult to maintain emulsion stability and causing a drop in viscosity and an increase in size of emulsion particles. To solve such problems, a large amount of thickener is used to prevent the coalescence of pearl particles dispersed in an outer phase and the attack to the emulsion interface. In this manner, it is possible to assist stable dispersion of pearl particles and stabilization of an emulsion formulation. However, in this case, the thickener causes a sticky feeling of use and soft spreadability may not be obtained due to high viscosity. In addition, use of a large amount of surfactant, thickener or thickener causes degradation of skin safety.

Spherical microparticles including polymers have a size and shape controllable depending on preparation methods thereof, and thus have high applicability. For example, there is provided Pickering emulsion which uses spherical microparticles to form stabilized macroemulsion particles. The contact angle (θ) between an aqueous phase and an oil phase varies with hydrophilicity/hydrophobicity of spherical particles. When a contact angle is larger than 90°, O/W emulsion particles are formed. Meanwhile, when a contact angle is smaller than 90°, W/0 emulsion particles are formed.

In addition, some attempts have been made to impart amphiphilic property (i.e. both hydrophilic property and hydrophobic property) to spherical microparticles so that novel anisotropic powder may be obtained. This may be exemplified by Janus spherical particles. However, such spherical particles have a limitation in chemical anisotropy due to their morphological limitation. In other words, although the particles are morphologically anisotropic, they may be hydrophobic or hydrophilic as a whole, and thus have limited chemical anisotropy.

Therefore, some attempts have been made to obtain surface active anisotropic powder by controlling a geometrical shape and imparting chemical anisotropy. However, no method for mass production of amphiphilic anisotropic powder has been developed to date, although such amphiphilic anisotropic powder shows high applicability. Moreover, it is difficult to produce amphiphilic anisotropic powder uniformly in a large amount in an industrial scale, leading to a failure in practical application.

### DISCLOSURE

### Technical Problem

A technical problem to be solved by the present disclosure is to provide a stable emulsion type cosmetic compound including a light interference pigment.

Another technical problem to be solved by the present disclosure is to provide an emulsion type cosmetic compound which has excellent skin safety.

Still another technical problem to be solved by the present disclosure is to provide an emulsion type cosmetic compound which includes a light interference pigment dispersed homogeneously therein without precipitation or coalescence and has excellent formulation and emulsion stability.

Still another technical problem to be solved by the present disclosure is to provide an emulsion type cosmetic composition which includes a light interference pigment dispersed homogeneously therein without precipitation or coalescence even in the case of a high content of the light interference pigment, and has excellent formulation and emulsion stability.

Still another technical problem to be solved by the present disclosure is to provide an emulsion type cosmetic compound which provides a stable formulation without using an excessive amount of thickener.

Still another technical problem to be solved by the present disclosure is to provide an emulsion type cosmetic compound which prevents skin irritation by avoiding the use of an excessive amount of thickener, dispersant or surfactant.

Still another technical problem to be solved by the present disclosure is to provide an emulsion type cosmetic compound which shows a fresh feeling of use and watery feeling by virtue of a watering effect of emulsion particles.

Yet another technical problem to be solved by the present disclosure is to provide an emulsion type cosmetic compound which provides a matte and powdery finishing feeling.

### Technical Solution

In one general aspect, there is provided an emulsion type cosmetic composition including amphiphilic anisotropic powder and a light interference pigment, wherein the amphiphilic anisotropic powder includes a first hydrophilic polymer spheroid and a second hydrophobic polymer spheroid, the first polymer spheroid and the second polymer spheroid are bound to each other with a structure in which one polymer spheroid at least partially penetrates into the other polymer spheroid, the first polymer spheroid has a core-shell structure, and the shell has a functional group, and the light interference pigment has an average particle diameter of 1-20 μm.

### Advantageous Effects

In one aspect, the present disclosure provides a stable emulsion type cosmetic compound including a light interference pigment.

In another aspect, the present disclosure provides an emulsion type cosmetic compound which has excellent skin safety.

In still another aspect, the present disclosure provides an emulsion type cosmetic compound which includes a light interference pigment dispersed homogeneously therein without precipitation or coalescence and has excellent formulation and emulsion stability.

In still another aspect, the present disclosure provides an emulsion type cosmetic composition which includes a light interference pigment dispersed homogeneously therein without precipitation or coalescence even in the case of a high content, and has excellent formulation and emulsion stability.

In still another aspect, the present disclosure provides an emulsion type cosmetic compound which provides a stable formulation without using an excessive amount of thickener.

In still another aspect, the present disclosure provides an emulsion type cosmetic compound which prevents skin irritation by avoiding the use of an excessive amount of thickener, dispersant or surfactant.

In still another aspect, the present disclosure provides an emulsion type cosmetic compound which shows a fresh feeling of use and watery feeling by virtue of a watering effect of emulsion particles.

In yet another aspect, the present disclosure provides an emulsion type cosmetic compound which provides a matte and powdery finishing feeling.

### DESCRIPTION OF DRAWINGS

FIG. 1 is a schematic view illustrating the formation of amphiphilic anisotropic powder according to an embodiment of the present disclosure.

FIG. 2 is a Table that shows electron microscopic images illustrating the emulsion particles of the compositions according to Example 1 and Comparative Examples 1-3 and particle sizes thereof, observed on the day of preparation and after the lapse of 1 week.

FIG. 3 is a graph illustrating a change in viscosity of each of the compositions according to Example 1 and Comparative Examples 1-3.

### BEST MODE

Exemplary embodiments now will be described more fully hereinafter with reference to the accompanying drawings, in which exemplary embodiments are shown. This disclosure may, however, be embodied in many different forms and should not be construed as limited to the exemplary embodiments set forth therein. Rather, these embodiments are provided so that this disclosure will be thorough and complete, and will fully convey the scope of the present disclosure to those skilled in the art. In the drawings, the shape, size and regions, and the like, of the drawing may be exaggerated for clarity. In addition, although a part of constitutional elements is shown for convenience of description, the remaining part may be understood with ease by those skilled in the art. Further, it will be understood by those skilled in the art that various changes in form and details may be made thereto without departing from the scope of this disclosure as defined by the appended claims.

As used herein, “substituted” means that at least one hydrogen atom of the functional group described herein is substituted with a halogen atom (F, Cl, Br or I), hydroxyl group, nitro group, imino group (═NH, ═NR, wherein R is a C1-C10 alkyl group), amidino group, hydrazine or hydrazone group, carboxyl group, substituted or non-substituted C1-C20 alkyl group, substituted or non-substituted C3-C30 heteroaryl group, or substituted or non-substituted C2-C30 heterocycloalkyl group, unless otherwise stated.

As used herein, “(meth)acryl” means acryl and/or methacryl.

As used herein, the particle size of amphiphilic anisotropic powder is measured as the maximum length that is the maximum length of the powder particles. As used herein, the particle size range of amphiphilic anisotropic powder means that at least 95% of the amphiphilic anisotropic powder present in a composition belongs to the corresponding range.

As used herein, the average particle diameter of emulsion particles means the average of diameter of each particle. As used herein, the average particle diameter range of emulsion particles means that at least 95% of the emulsion particles present in a composition belongs to the corresponding range.

As used herein, the average particle diameter of light interference pigment means the volumetric average particle diameter obtained by calculating the volumetric average based on the particle size distribution determined by the known methods for determining a particle size distribution, such as observation of electron microscopic images, laser diffraction, or the like.

In one aspect, there is provided an emulsion type cosmetic composition including a light interference pigment and amphiphilic anisotropic powder. The composition may be used as a makeup cosmetic composition including a light interference pigment for imparting a pearl-like effect.

As used herein, ‘light interference pigment’ is also called a pearl pigment or pearlescent pigment, shows a pearlescent gloss, rainbow-like light or metal-like gloss through an interference phenomenon caused by the light reflected on the pigment surface, and has a plate-like or needle-like powdery shape. It has been used mainly for imparting a pearl-like effect or pearl-like color to a makeup cosmetic compound.

The light interference pigment may have an average particle diameter of 0.5-30 μm, particularly 1-20 μm, and more particularly 2-16 μm. For example, the light interference pigment may have an average particle diameter of 0.5 μm or more, 0.6 μm or more, 0.8 μm or more, 0.9 μm or more, 1 μm or more, 1.5 μm or more, 2 μm or more, 2.5 μm or more, 3 μm or more, 3.5 μm or more, 4 μm or more, 4.5 μm or more, or 5 μm or more; and 30 μm or less, 29 μm or less, 27 μm or less, 26 μm or less, 25 μm or less, 24 μm or less, 23 μm or less, 22 μm or less, 21 μm or less, 20 μm or less, 19 μm or less, 18 μm or less, 17 μm or less, or 16 μm or less. The composition according to an embodiment of the present disclosure may include a pigment having a relatively large size stably in its formulation without precipitation or coalescence.

According to another embodiment, the light interference pigment may be used in an amount of 0.1 wt % or more, 0.2 wt % or more, 0.3 wt % or more, 0.4 wt % or more, 0.5 wt % or more, 0.6 wt % or more, 0.7 wt % or more, 0.8 wt % or more, 0.9 wt % or more, 1.0 wt % or more, 1.1 wt % or more, 1.2 wt % or more, 1.3 wt % or more, 1.4 wt % or more, 1.5 wt % or more, 1.6 wt % or more, 1.7 wt % or more, 1.8 wt % or more, 1.9 wt % or more, or 2.0 wt % or more; and 10 wt % or less, 9.5 wt % or less, 9.0 wt % or less, 8.5 wt % or less, 8.0 wt % or less, 7.5 wt % or less, 7.0 wt % or less, 6.5 wt % or less, 6.0 wt % or less, 5.5 wt % or less, 5.0 wt % or less, 4.5 wt % or less, 4.0 wt % or less, 3.5 wt % or less, or 3.0 wt % or less, based on the total weight of the composition. For example, the light interference pigment may be used in an amount of 0.1 wt %-10 wt %, 0.5 wt %-5 wt %, or 1 wt %-4 wt %, based on the total weight of the composition. It is possible to provide an excellent pearl-like effect and to maintain a stable emulsion formulation within the above-defined range. The composition according to an embodiment of the present disclosure maintains a stable emulsion state even in the presence of such a high content of pigment, and to maintain a state in which the pigment is dispersed homogeneously in the composition without coalescence of pigment particles.

According to still another embodiment, the light interference pigment may include lead carbonate, BiOCl, TiO₂-coated mica, TiO₂-coated synthetic mica, TiO₂-coated aluminum oxide (Al₂O₃), TiO₂-coated silicon oxide (SiO₂), glass flake, or the like, but is not limited thereto.

According to an embodiment of the present disclosure, the emulsion interface of emulsion particles is maintained firmly by virtue of the amphiphilic anisotropic powder. In addition, it is possible to provide a soft feeling of use without coalescence of light interference pigment particles.

According to still another embodiment, the amphiphilic anisotropic powder includes a first hydrophilic polymer spheroid and a second hydrophobic polymer spheroid, wherein the first polymer spheroid and the second polymer spheroid are bound to each other with a structure in which one polymer spheroid at least partially penetrates into the other polymer spheroid, the first polymer spheroid has a core-shell structure, and the shell has a functional group.

As used herein, a spheroid means a single body formed of polymers. For example, it may have a spherical, globoidal or oval shape and a micro-scale or nano-scale long axis length based on the largest length in the section of the body.

According to an embodiment, the second polymer spheroid and the core of the first polymer spheroid may include vinyl polymers, and the shell of the first polymer spheroid may include a copolymer of a vinyl monomer with a functional group-containing monomer.

According to another embodiment, the vinyl polymer may include a vinyl aromatic polymer, particularly polystyrene.

According to still another embodiment, the vinyl monomer may include a vinyl aromatic monomer. For example, the vinyl monomer may be substituted or non-substituted styrene.

According to still another embodiment, the functional group may be siloxane.

According to still another embodiment, the functional group-containing monomer may be a siloxane-containing (meth)acrylate, particularly at least one selected from the group consisting of 3-(trimethoxysilyl)propyl acrylate, 3-(trimethoxysilyl) propyl methacrylate, vinyltriethoxysilane and vinyltrimethoxysilane, or a combination thereof.

According to still another embodiment, the shell of the polymer spheroid may further have a hydrophilic functional group introduced thereto.

According to still another embodiment, the hydrophilic functional group may be a negatively charged or positively charged functional group or polyethylene glycol (PEG)-based functional group and may include at least one selected from the group consisting of a carboxylate group, sulfone group, phosphate group, amino group, alkoxy group, ester group, acetate group, polyethylene glycol group and hydroxyl group.

According to still another embodiment, the shell of the first polymer spheroid may further have a saccharide-containing functional group introduced thereto.

According to still another embodiment, the saccharide-containing functional group may be derived from at least one selected from the group consisting of N—{N-(3-triethoxysilylpropyl)aminoethyl}gluconamide, N-(3-triethoxysilylpropyl) gluconamide and N—{N-(3-triethoxysilylpropyl)aminoethyl}-oligo-hyaluronamide.

According to still another embodiment, the amphiphilic anisotropic powder may have a symmetric shape, asymmetric snowman shape or asymmetric reverse snowman shape on the basis of the binding portion where the first polymer spheroid and the second polymer spheroid are bound to each other. The snowman shape refers to the first polymer spheroid and the second polymer spheroid bound to each other and having a different size.

According to still another embodiment, the amphiphilic anisotropic powder may have a particle size of 100-2500 nm. In a variant, the amphiphilic anisotropic powder may have a particle size of 100-1500 nm, 100-500 nm, or 200-300 nm. Particularly, the amphiphilic powder may have a particle size of 100 nm or more, 200 nm or more, 300 nm or more, 400 nm or more, 500 nm or more, 600 nm or more, 700 nm or more, 800 nm or more, 900 nm or more, 1000 nm or more, 1100 nm or more, 1200 nm or more, 1300 nm or more, 1400 nm or more, or 1500 nm or more; and 2500 nm or less, 2400 nm or less, 2300 nm or less, 2200 nm or less, 2100 nm or less, 2000 nm or less, 1900 nm or less, 1800 nm or less, 1700 nm or less, 1600 nm or less, 1500 nm or less, 1400 nm or less, 1300 nm or less, 1200 nm or less, 1100 nm or less, 1000 nm or less, 900 nm or less, 800 nm or less, 700 nm or less, 600 nm or less, 500 nm or less, 400 nm or less, 300 nm or less, or 200 nm or less.

According to still another embodiment, the amphiphilic anisotropic powder may form macroemulsion particles having a size of 2-500 μm. In a variant, the amphiphilic anisotropic powder may form macroemulsion particles having a size of 5-400 μm, 10-350 μm, 30-300 μm, or 50-300 μm. Particularly, the amphiphilic anisotropic powder may form emulsion particles having a size of 2 μm or more, 3 μm or more, 4 μm or more, 5 μm or more, 6 μm or more, 7 μm or more, 8 μm or more, 9 μm or more, 10 μm or more, 11 μm or more, 12 μm or more, 13 μm or more, 14 μm or more, 15 μm or more, 16 μm or more, 17 μm or more, 18 μm or more, 19 μm or more, 20 μm or more, 21 μm or more, 22 μm or more, 23 μm or more, 24 μm or more, 25 μm or more, 26 μm or more, 27 μm or more, 28 μm or more, 29 μm or more, 30 μm or more, 31 μm or more, 32 μm or more, 33 μm or more, 34 μm or more, 35 μm or more, 36 μm or more, 37 μm or more, 38 μm or more, 39 μm or more, 40 μm or more, 41 μm or more, 42 μm or more, 43 μm or more, 44 μm or more, 45 μm or more, 46 μm or more, 47 μm or more, 48 μm or more, 49 μm or more, or 50 μm or more; and 500 μm or less, 490 μm or less, 480 μm or less, 470 μm or less, 460 μm or less, 450 μm or less, 440 μm or less, 430 μm or less, 420 μm or less, 410 μm or less, 400 μm or less, 390 μm or less, 380 μm or less, 370 μm or less, 360 μm or less, 350 μm or less, 340 μm or less, 330 μm or less, 320 μm or less, 310 μm or less, 300 μm or less, 290 μm or less, 280 μm or less, 270 μm or less, 260 μm or less, 250 μm or less, 240 μm or less, 230 μm or less, 220 μm or less, 210 μm or less, 200 μm or less, 190 μm or less, 180 μm or less, 170 μm or less, 160 μm or less, or 150 μm or less.

Since the hydrophobic part and hydrophilic part of the amphiphilic anisotropic powder have different orientability against the interface, it is possible to form macroemulsion particles. It is possible to provide an emulsion formulation having various viscosities, including a formulation having a less viscous soft feeling of use, by virtue of such macroemulsion particles.

While an interface film formed by a conventional molecular-level surfactant forms a dynamic emulsion phase, the thickness of the interface film of the emulsion particles formed by the amphiphilic anisotropic powder increases to several hundreds of nanometers and a stabilized interface film is formed by virtue of the strong binding among the powder particles. Therefore, it is possible to maintain a stable emulsion state, while not being affected by the light interference pigment.

According to still another embodiment, the amphiphilic anisotropic powder may be present in an amount of 0.1 wt % or more, 0.2 wt % or more, 0.3 wt % or more, 0.4 wt % or more, 0.5 wt % or more, 0.6 wt % or more, 0.7 wt % or more, 0.8 wt % or more, 0.9 wt % or more, or 1.0 wt % or more; and 30 wt % or less, 29 wt % or less, 28 wt % or less, 27 wt % or less, 26 wt % or less, 25 wt % or less, 24 wt % or less, 23 wt % or less, 22 wt % or less, 21 wt % or less, 20 wt % or less, 19 wt % or less, 18 wt % or less, 17 wt % or less, 16 wt % or less, 15 wt % or less, 14 wt % or less, 13 wt % or less, 12 wt % or less, 11 wt % or less, 10 wt % or less, 9 wt % or less, 8 wt % or less, 7 wt % or less, 6 wt % or less, 5 wt % or less, 4 wt % or less, or 3 wt % or less, based on the total weight of the composition. For example, the amphiphilic anisotropic powder may be present in an amount of 0.1-30 wt %, particularly 0.5-20 wt %, more particularly 1-10 wt %, and even more particularly 1-3 wt %, based on the total weight of the composition. It is possible to form stable emulsion particles and to form emulsion particles having an adequate size within the above-defined range.

The composition may have a viscosity of 1000 cps or more, 1100 cps or more, 1200 cps or more, 1300 cps or more, 1400 cps or more, 1500 cps or more, 1600 cps or more, 1700 cps or more, 1800 cps or more, 1900 cps or more, 2000 cps or more, 2100 cps or more, 2200 cps or more, 2300 cps or more, 2400 cps or more, or 2500 cps or more; and 30000 cps or less, 29000 cps or less, 28000 cps or less, 27000 cps or less, 26000 cps or less, 25000 cps or less, 24000 cps or less, 23000 cps or less, 22000 cps or less, 21000 cps or less, 20000 cps or less, 19000 cps or less, 18000 cps or less, 17000 cps or less, 16000 cps or less, 15000 cps or less, 14000 cps or less, 13000 cps or less, 12000 cps or less, 11000 cps or less, 10000 cps or less, 8900 cps or less, 8800 cps or less, 8700 cps or less, 8600 cps or less, 8500 cps or less, 8400 cps or less, 8300 cps or less, 8200 cps or less, 8100 cps or less, 8000 cps or less, 7900 cps or less, 7800 cps or less, 7700 cps or less, 7600 cps or less, 7500 cps or less, 7400 cps or less, 7300 cps or less, 7200 cps or less, 7100 cps or less, 7000 cps or less, 6900 cps or less, 6800 cps or less, 6700 cps or less, 6600 cps or less, 6500 cps or less, 6400 cps or less, 6300 cps or less, 6200 cps or less, 6100 cps or less, or 6000 cps or less. For example, the viscosity may be 1000-30000 cps, 1000-20000 cps, 1500-10000 cps, or 2000-7000 cps. The composition may form macroemulsion particles having a firm emulsion interface. In addition, the single amphiphilic anisotropic powder that does not form emulsion particles is present. Thus, it is possible for the light interference pigment to be dispersed homogeneously without precipitation or coalescence. Further, the formulation may have excellent emulsion stability.

The composition according to the present disclosure maintains the stability of an emulsion formulation without using an additional thickener or wax, even when it includes a high content of light interference pigment. Therefore, it is possible to provide a composition having a broad range of viscosities as mentioned above. Even when a high content of light interference pigment is used, it is possible to provide a composition with a low viscosity of 8000 cps or less, 7000 cps or less, or 4000-7000 cps. It is possible to provide a flowable soft formulation within the above-defined range, and thus to show a non-sticky fresh feeling of use.

The cosmetic composition according to an embodiment of the present disclosure may be obtained by the method which includes preparing the amphiphilic anisotropic powder, and emulsifying an oil phase part and an aqueous phase part by using the amphiphilic anisotropic powder.

According to an embodiment, the amphiphilic anisotropic powder may be obtained by the method, including: polymerizing a first monomer to obtain a core of a first polymer spheroid; coating the core of the first polymer spheroid to obtain a first polymer spheroid having a core-shell structure; and reacting the first polymer spheroid having a core-shell structure with a first monomer to obtain amphiphilic anisotropic powder in which a second polymer spheroid is formed.

FIG. 1 is a schematic view illustrating formation of the amphiphilic anisotropic powder according to an embodiment of the present disclosure. It is possible to form a second polymer spheroid by allowing the core of the first polymer spheroid to penetrate through the shell of the first polymer spheroid and to grow toward the exterior by using the above-mentioned method.

According to another embodiment, the method for preparing amphiphilic anisotropic powder may include: (1) agitating a first monomer and a polymerization initiator to form a core of a first polymer spheroid; (2) agitating the formed core of a first polymer spheroid with a first monomer, a polymerization initiator and a functional group-containing monomer to form a first polymer spheroid having a coated core-shell structure; and (3) agitating the formed first polymer spheroid having a core-shell structure with a second monomer and a polymerization initiator to obtain anisotropic powder in which a second polymer spheroid is formed.

In steps (1), (2) and (3), the agitation may be rotary agitation. Since homogeneous mechanical mixing is required together with chemical modification in order to produce uniform particles, rotary agitation is preferred. The rotary agitation may be carried out in a cylindrical reactor but is not limited thereto.

Herein, the internal design of the reactor significantly affects powder formation. The size and position of the baffles of the cylindrical reactor and the distance from an impeller have a significant effect upon the uniformity of the particles to be produced. Preferably, the interval between the internal baffle and the blade of an impeller is minimized to make convection flow and intensity thereof uniform, the powdery reaction mixture is introduced to a level lower than the baffle length, and the impeller is maintained at a high rotation speed. The rotation speed may be 200 rpm or higher, and the ratio of the diameter to the height of the reactor may be 1-3:1-5. Particularly, the reactor may have a diameter of 10-30 cm and a height of 10-50 cm. The reactor may have a size variable in proportion to the reaction capacity. In addition, the cylindrical reactor may be made of ceramics, glass or the like. The agitation is carried out preferably at a temperature of 50-90° C.

Simple mixing in a cylindrical rotary reactor allows production of uniform particles, requires low energy consumption and provides maximized reaction efficiency, and thus is amenable to mass production. The conventional tumbling method including rotation of a reactor itself causes inclination of the whole part of the reactor with a certain angle and rotation at a high speed, and thus requires high energy consumption and limits the reactor size. Due to such limitation in reactor size, the output is limited to a small amount of approximately several tens of milligrams to several grams. Thus, the conventional tumbling method is not suitable for mass production.

According to an embodiment, the first monomer and the second monomer may be the same or different, and particularly may be a vinyl monomer. In addition, the first monomer added in step (2) may be the same as the first monomer used in step (1) and the polymerization initiator used in each step may be the same or different.

According to another embodiment, the vinyl monomer may be a vinyl aromatic monomer. The vinyl aromatic monomer may be substituted or non-substituted styrene.

According to still another embodiment, the polymerization initiator may be a radical polymerization initiator. Particularly, the polymerization initiator may be a peroxide-based or azo-based initiator, or a combination thereof. In addition, ammonium persulfate, sodium persulfate or potassium persulfate may be used.

According to still another embodiment, in step (1), the first monomer and the polymerization initiator may be mixed at a weight ratio of 100-1000:1. In a variant, the first monomer and the polymerization initiator may be mixed at a weight ratio of 100-750:1, 100-500:1, or 100-250:1.

In a variant, in step (1), a stabilizer is added together with the first monomer and the polymerization initiator in such a manner that the first monomer, the polymerization initiator and the stabilizer may be mixed at a weight ratio of 100-1000:1:0.001-5. The size and shape of the powder is determined by controlling the size of the first polymer spheroid in step (1), and the size of the first polymer spheroid may be controlled by the ratio of the first monomer, the polymerization initiator and the stabilizer. In addition, it is possible to increase the uniformity of anisotropic powder in its size and shape by mixing the first monomer, the polymerization initiator and the stabilizer within the above-defined ratio.

According to an embodiment, the stabilizer may be an ionic vinyl monomer, and particularly sodium 4-vinylbenzene sulfonate may be used. The stabilizer prevents swelling of the particles, and imparts positive or negative charges to the powder surface, thereby preventing coalescence (binding) of the particles electrostatically.

When the amphiphilic anisotropic powder has a size of 200-250 nm, it may be obtained from the first polymer spheroid including the first monomer, the polymerization initiator and the stabilizer at a ratio of 80-135:1:1-5, particularly 95-120:1:2-4.

In addition, when the amphiphilic anisotropic powder has a size of 400-450 nm, it may be obtained from the first polymer spheroid including the first monomer, the polymerization initiator and the stabilizer at a ratio of 225-240:1:1-3, particularly 230-235:1:1-3.

Further, when the amphiphilic anisotropic powder has a size of 1100-2500 nm, it may be obtained from the first polymer spheroid prepared by reacting the first monomer, the polymerization initiator and the stabilizer at a ratio of 110-130:1:0, particularly 115-125:1:0.

In addition, amphiphilic anisotropic powder having an asymmetric snowman shape may be obtained from the first polymer spheroid prepared by reacting the first monomer, the polymerization initiator and the stabilizer at a ratio of 100-140:1:8-12, particularly 110-130:1:9-11.

Further, amphiphilic anisotropic powder having an asymmetric reverse snowman shape may be obtained from the first polymer spheroid prepared by reacting the first monomer, the polymerization initiator and the stabilizer at a ratio of 100-140:1:1-5; particularly 110-130:1:2-4.

According to still another embodiment, the functional group-containing monomer in step (2) may be a siloxane-containing (meth)acrylate, such as 3-(trimethoxysilyl)propyl acrylate, 3-(trimethoxysilyl)propyl methacrylate, vinyl triethoxysilane, vinyl trimethoxysilane or a combination thereof.

According to still another embodiment, in step (2), the first monomer, the polymerization initiator and the functional group-containing compound may be mixed at a weight ratio of 30-100:0.2-1.0:1-20. In a variant, the first monomer, the polymerization initiator and the functional group-containing compound may be mixed at a weight ratio of 150-300:1:6-40. It is possible to control the coating degree according to the reaction ratio, and then the coating degree determines the shape of amphiphilic anisotropic powder. When the first monomer, the polymerization initiator and the functional group-containing compound are used within the above-defined ratio, the coating thickness is increased by about 10-30%, particularly approximately 20%, based on the initial thickness. In this case, formation of powder proceeds smoothly without problems, such as a failure in formation of powder caused by excessively thick coating or multi-directional formation of powder caused by excessively thin coating. In addition, it is possible to increase the uniformity of anisotropic powder within the above weight ratio.

In step (3), the core of the first polymer spheroid penetrates through the shell from one direction of the first polymer spheroid having a core-shell structure and protrudes out from the shell. Then, the protrusion may be grown by the polymer of the second monomer to form a shape of anisotropic powder.

According to still another embodiment, in step (3), the second monomer and the polymerization initiator may be mixed at a weight ratio of 150-250:1. In a variant, the second monomer and the polymerization initiator may be mixed at a weight ratio of 160-250:1, 170-250:1, 180-250:1, 190-250:1, 200-250:1, 210-250:1, 220-250:1, 230-250:1, or 240-250:1.

In a variant, in step (3), a stabilizer may be added together with the second monomer and the polymerization initiator in such a manner that the second monomer, the polymerization initiator and the stabilizer may be mixed at a weight ratio of 150-250:1:0.001-5. Particular examples of the stabilizer are the same as described above. It is possible to increase the uniformity of anisotropic powder within the above-defined weight ratio.

According to still another embodiment, in step (3), the second monomer may be mixed in an amount of 40-300 parts by weight based on 100 parts by weight of the first polymer spheroid having a core-shell structure. Particularly, when the content of the second monomer is 40-100 parts by weight based on 100 parts by weight of the first polymer spheroid, asymmetric snowman-like powder is obtained. When the content of the second monomer is 100-150 parts by weight or 110-150 parts by weight, symmetric powder is obtained. In addition, when the content of the second monomer is 150-300 parts by weight or 160-300 parts by weight, asymmetric reverse snowman-like powder is obtained. It is possible to increase the uniformity of anisotropic powder within the above weight ratio.

According to still another embodiment, the method for preparing amphiphilic anisotropic powder may further include, after step (3), step (4) of introducing a hydrophilic functional group to the anisotropic powder.

According to still another embodiment, the hydrophilic functional group in step (4) may be introduced by using a silane coupling agent and a reaction modifier, but is not limited thereto.

According to still another embodiment, the silane coupling agent may be at least one selected from the group consisting of N-[(3-(trimethoxysilyl)propyl)ethylenediamine, N-[3-(trimethoxysilyl)propyl]ethylene diammonium chloride, (N-succinyl-3-aminopropyl)trimethoxysilane, 1-[3-(trimethoxysilyl)propyl]urea and 3-[(trimethoxysilyl)propyloxy]-1,2-propanediol. Particularly, the silane coupling agent may be N-[(3-(trimethoxysilyl) propyl)ethylenediamine.

According to still another embodiment, the silane coupling agent may be mixed in an amount of 35-65 parts by weight, particularly 40-60 parts by weight, based on 100 parts by weight of the anisotropic powder obtained from step (3). It is possible to carry out hydrophilization adequately within the above-defined range.

According to still another embodiment, the reaction modifier may be ammonium hydroxide.

According to still another embodiment, the reaction modifier may be mixed in an amount of 85-115 parts by weight, particularly 90-110 parts by weight, based on 100 parts by weight of the anisotropic powder obtained from step (3). It is possible to carry out hydrophilization adequately within the above-defined range.

According to still another embodiment, the method for preparing amphiphilic anisotropic powder may further include step (4) of introducing a saccharide-containing functional group to the anisotropic powder, after step (3).

In step (4), the saccharide-containing functional group may be introduced by using a saccharide-containing silane coupling agent and a reaction modifier, but is not limited thereto.

According to still another embodiment, the saccharide-containing silane coupling agent may be at least one selected from the group consisting of N—{N-(3-triethoxysilylpropyl)aminoethyl}gluconamide, N-(3-triethoxysilylpropyl) gluconamide and N—{N-(3-triethoxysilylpropyl)aminoethyl}-oligo-hyaluronamide.

According to still another embodiment, the reaction modifier may be ammonium hydroxide.

According to still another embodiment, the reaction modifier may be mixed in an amount of 85-115 parts by weight, particularly 90-110 parts by weight, based on 100 parts by weight of the anisotropic powder obtained from step (3). It is possible to introduce the saccharide-containing functional group adequately within the above-defined range.

The method for preparing amphiphilic anisotropic powder disclosed herein uses no crosslinking agent, thereby causing no agglomeration and providing high yield and uniformity. In addition, the method disclosed herein uses a simple agitation process and is more amenable to mass production as compared to a tumbling process. Particularly, the method disclosed herein is advantageous in that it allows production of nano-size particles having a size of 300 nm or less in a large scale of several tens of grams and several tens of kilograms.

According to still another embodiment, the emulsion composition may be an oil-in-water type or water-in-oil type emulsion composition, particularly an oil-in-water type emulsion composition.

When the composition is an oil-in-water type composition, the light interference pigment is present in the inner phase or outer phase. The light interference pigment may be present in the outer phase and dispersed stably without precipitation or coalescence of pigment particles as described above. In addition, the light interference pigment may be present in the inner phase and in the emulsion particles, and may be dispersed stably by virtue of a firm emulsion interface which prevents coalescence of emulsion particles.

According to an embodiment, the composition may form a formulation having a characteristic feeling of use resulting from a watery effect of macropowder emulsion particles, not a conventional water-in-oil type viscous/rigid formulation. The composition according to an embodiment of the present disclosure allows the light interference pigment to be dispersed homogeneously without using a high content of thickener, and thus prevents stickiness or skin irritation caused by the thickener.

The composition according to an embodiment of the present disclosure undergoes a collapse of formulation with ease upon the skin application to provide soft spreadability.

The composition according to an embodiment of the present disclosure prevents skin irritation that may occur due to the addition of a dispersant or an excessive amount of surfactant.

The composition according to an embodiment of the present disclosure shows excellent emulsion stability even though it contains a light interference pigment, and thus may provide a soft feeling of use as an emulsion composition simultaneously with a fresh feeling and watery feeling derived from the watering effect. Particularly, the composition shows the above-mentioned effects while the stability thereof is not affected even in the presence of a high content of light interference pigment.

The composition according to an embodiment of the present disclosure avoids a sticky finishing feeling caused by a surfactant, and provides a matte and powdery finishing feeling by virtue of the presence of independent amphiphilic anisotropic powder that does not form emulsion particles.

The composition according to an embodiment of the present disclosure shows emulsion stability with time over a broad range of temperatures, such as a temperature ranging from −15° C. to 60° C., particularly from −10° C. to 55° C.

The composition according to an embodiment of the present disclosure includes macroemulsion particles to provide a soft and silky feeling of use.

In the composition according to an embodiment of the present disclosure, a watering effect occurs to such a degree that it may be seen by the naked eyes, while the inner phase of the macroemulsion particles are ejected right upon the application. In this manner, the composition supplies water to the skin, shows an effect of trimming the skin texture, imparts a pearl-like effect to the skin by the light interference pigment, realizes a gorgeous skin tone, provides a matte and close contact feeling by virtue of the powdery and light feeling of use derived from the amphiphilic anisotropic powder, and maintains persistency.

The composition according to an embodiment of the present disclosure may maintain emulsion formulation stability even when it additionally includes an excessive amount of ethanol or salt in order to provide a formulation with a fresh feeling.

The composition according to an embodiment of the present disclosure may be formulated by incorporating a cosmetically or dermatologically acceptable medium or base thereto. Such a formulation includes any formulation suitable for local application, and may be provided in the form of suspension, microemulsion, microcapsules, microgranules or ionic (liposome) and non-ionic vesicular dispersant, or in the form of cream, skin, lotion, powder, ointment, spray or conceal stick. In addition, the composition may be used in the form of foam or an aerosol composition further including a pressurized propellant. Such compositions may be obtained by the methods known to those skilled in the art.

The composition according to an embodiment of the present disclosure may be formulated into various formulations for makeup, including foundation, liquid foundation, concealer and makeup base, but is not limited thereto.

In addition, the composition according to an embodiment of the present disclosure may include supplementary ingredients conventionally used in the field of cosmetics or dermatology, such as powder, a fat material, organic solvent, solubilizer, concentrating agent, gelling agent, softening agent, anti-oxidant, suspending agent, stabilizer, foaming agent, perfuming agent, surfactant, water, ionic or non-ionic emulsifier, filler, metal ion chelator, chelating agent, preservative, vitamin, protector, wetting agent, essential oil, dye, pigment, hydrophilic or oleophilic activating agent, lipid vesicles or any other ingredients conventionally used for cosmetics. Such supplementary ingredients are introduced in an amount used generally in the field of cosmetics or dermatology. The composition according to an embodiment of the present disclosure may further include a skin absorption enhancer to increase the effect of improving skin conditions.

### Modes for Invention

The examples will now be described to illustrate the present disclosure in detail. It will be appreciated by those skilled in the art that the following examples are for illustrative purposes only and not intended to limit the scope of the present disclosure.

### Preparation Examples 1-4

Preparation Examples 1-4 are obtained according to the compositions of the following Table 1. The preparation method will be explained hereinafter.

The ingredients used for Preparation Examples 1-4 are shown below.

TABLE 1

| PS | | | | | |
|---|---|---|---|---|---|
| (1 L shaking type reaction tank) | | CS | | DB | |
| Water | 300 | PS | 300 | CS | 240 |
| MeOH | 40 | Water | 250 | Water | 350 |
| Styrene | 50 | TMSPA | 6 | AIBN | 0.2 |
| KPS | 0.5 | Styrene | 50 | Styrene | 40 |
| SVBS | 1.0 | AIBN | 0.2 | SVBS | 0.35 |

MeOH: Methanol (cosolvent)
KPS: Potassium persulfate (initiator)
SVBS: Sodium vinyl benzene sulfonate (stabilizer)
PS: Polystyrene (polymer beads)
CS: Coated first polymer spheroid having a core-shell structure
DB: amphiphilic anisotropic powder
TMSPA: Trimethoxysilyl propylacrylate (functional group)
AIBN: Azobisisobutyronitrile (polymerization initiator)

### Preparation Example 1. Preparation of Polystyrene (PS) First Polymer Spheroid

First, 50 g of styrene as a monomer, 1.0 g of sodium 4-vinylbenzene sulfonate as a stabilizer and 0.5 g of azobisisobutyronitrile (AIBN) as a polymerization initiator are mixed in an aqueous phase and are allowed to react at 75° C. for 8 hours. The reaction is carried out by agitating the reaction mixture in a cylindrical reactor having a diameter of 11 cm and a height of 17 cm and made of glass under a speed of 200 rpm.

### Preparation Example 2. Preparation of Coated First Polymer Spheroid Having Core-Shell (CS) Structure

First, 300 g of the polystyrene (PS) first polymer spherical particles obtained as described above are mixed with 50 g of styrene as a monomer, 6 g of 3-(trimethoxysilyl)propyl acrylate (TMSPA) and 0.2 g of azobisisobutyronitrile (AIBN) as a polymerization initiator and the reaction mixture is allowed to react at 75° C. for 8 hours. The reaction is carried out by agitating the reaction mixture in a cylindrical reactor.

### Preparation Example 3. Preparation of Amphiphilic Anisotropic Powder (DB)

First, 240 g of the aqueous dispersion of the polystyrene-core shell (PC-CS) dispersion obtained as described above is mixed with 40 g of styrene as a monomer, 0.35 g of sodium 4-vinylbenzene sulfonate as a stabilizer and 0.2 g of azobisisobutyronitrile (AIBN) as a polymerization initiator and the reaction mixture is heated to 75° C. to carry out reaction for 8 hours. The reaction is carried out by agitating the reaction mixture in a cylindrical reactor. In this manner, amphiphilic anisotropic powder having average particle size of 235 μm is obtained.

### Preparation Example 4. Preparation of Hydrophilized Amphiphilic Anisotropic Powder

First, 600 g of the aqueous dispersion of the anisotropic powder obtained as described above is mixed with 30 g of N-[3-(trimethoxysilyl)propyl]ethylenediamine) as a silane coupling agent and 60 g of ammonium hydroxide as a reaction modifier, and the reaction mixture is allowed to react at 25° C. for 24 hours to introduce a hydrophilic functional group. The reaction is carried out by agitating the reaction mixture in a cylindrical reactor to obtain hydrophilized amphiphilic anisotropic powder.

### [Example and Comparative Examples] Preparation of Emulsion Type Compositions

The oil-in-water type emulsion compositions of Example 1 and Comparative Examples 1-3 are prepared according to the compositions of the following Table 2. The ingredients used for the following Example and Comparative Examples are shown below.

(a) Oil: Butylene hydrogenated polydecene (Puresyn 4, Exxon Mobile)

(b) Surfactant: Glyceryl stearate/PEG-100 stearate (Arlacel 170-PA-(SG), Uniquema)

(c) Silicone oil: Decamethyl cyclopentasiloxane (DC345, Dow Corning)

(d) Water-dispersed amphiphilic anisotropic powder: Solution obtained by dissolving 10 wt % of the amphiphilic anisotropic powder of Preparation Example 3 in 55 wt % of purified water and 35 wt % of butylene glycol.

(e) Light interference pigment: Titanium dioxide/tin oxide/synthetic fluorphilogopite (cosmetic soft focus white 9003F, CQV, average particle diameter 2-16 μm)

(f) Ion chelator: Disodium EDTA (E.D.T.A.-2NA, Neord Co., Ltd.)

(g) Thickener: Polyacrylate-13 & polyisobutene & polysorbate 20 (Sepiplus 400, SEPPIC)

TABLE 2

| | Comp. | Comp. | Comp. | |
|---|---|---|---|---|
| (Unit: wt %) | Ex. 1 | Ex. 2 | Ex. 3 | Ex. 1 |
| Hydrogenated polydecene | 6 | 6 | 6 | 6 |
| Glyceryl stearate/ | 4 | 4 | | |
| PEG-100 stearate | | | | |
| Decamethyl | 6 | 6 | 6 | 6 |
| cyclopentasiloxane | | | | |
| Deionized water | 82.98 | 80.98 | 76.98 | 74.98 |
| Disodium EDTA | 0.02 | 0.02 | 0.02 | 0.02 |
| Water-dispersed amphiphilic | | | 10 | 10 |
| anisotropic powder | | | | |
| Light interference pigment | | 2 | | 2 |
| Polyacrylate-13 & | 1 | 1 | 1 | 1 |
| polyisobutene & | | | | |
| polysorbate 20 | | | | |

### [Test Example 1] Evaluation of Stability of Emulsion Particles

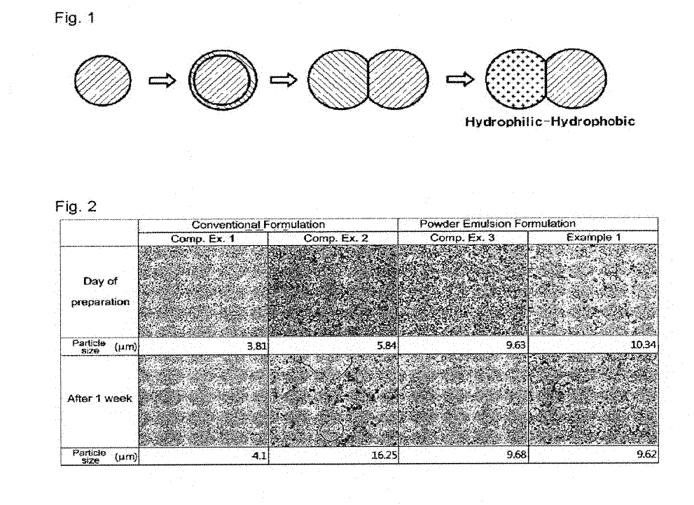

The compositions according to Example 1 and Comparative Examples 1-3 are allowed to stand at 30° C. for 1 week to evaluate the stability of emulsion particles. Each composition is photographed with an electron microscope on the day of preparation and 1 week after the preparation to observe a change in emulsion particles, and the particle size is determined. The results are shown in FIG. 2 in tabular form.

It can be seen from the results of FIG. 2 that while Comparative Example 1 using a conventional surfactant forms small emulsion particles on the day of its preparation, Comparative Example 2 including a light interference pigment in addition to a conventional surfactant system shows emulsion particles having a size increased by about 2 μm as compared to Comparative Example 1 on the day of its preparation, which suggests that the light interference pigment causes instability of the emulsion particles. After 1 week, Comparative Example 2 causes coalescence of the emulsion particles and shows an instable formulation state. On the contrary, Example 1 forms macroemulsion particles and causes no significant change in size of the emulsion particles, like Comparative Example 1 including no light interference pigment, even after the lapse of 1 week.

### [Test Example 2] Evaluation of Change in Viscosity

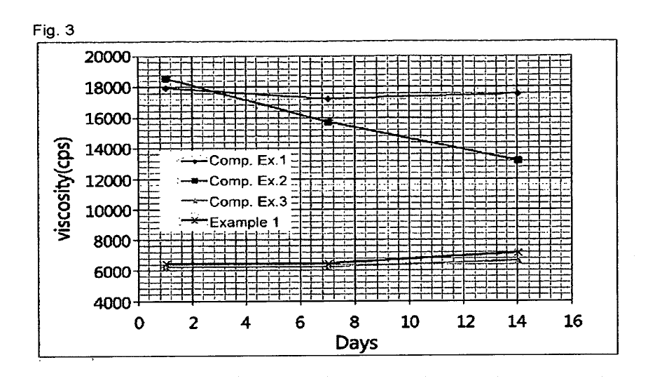

The viscosity of each of the compositions according to Example 1 and Comparative Examples 1-3 is measured by using Viscometer (LVDV-II+PRO, BROOKFIELD, USA), while being allowed to stand at 30° C. for 4 weeks. The results are shown in FIG. 3.

It can be seen from the results of FIG. 3 that Example 1 maintains a stable formulation with no change in viscosity, even though it has low viscosity like a formulation including no light interference pigment. On the contrary, Comparative Example 2 using the conventional surfactant system to which the light interference pigment is added shows a significant drop in viscosity. This demonstrates that the light interference pigment makes the emulsion formulation instable.

1. An emulsion type cosmetic composition comprising amphiphilic anisotropic powder and a light interference pigment,
wherein the amphiphilic anisotropic powder comprises a first hydrophilic polymer spheroid and a second hydrophobic polymer spheroid,
the first polymer spheroid and the second polymer spheroid are bound to each other with a structure in which one polymer spheroid at least partially penetrates into the other polymer spheroid,
the first polymer spheroid has a core-shell structure, and the shell has a functional group, and
the light interference pigment has an average particle diameter of 1-20 μm.

2. The emulsion type cosmetic composition according to claim 1, wherein the light interference pigment is present in an amount of 0.5-5 wt % based on the total weight of the composition.

3. The emulsion type cosmetic composition according to claim 1, wherein the light interference pigment comprises at least one selected from the group consisting of lead carbonate, BiOCl, TiO₂-coated mica, TiO₂-coated synthetic mica, TiO₂-coated aluminum oxide (Al₂O₃), TiO₂-coated silicon oxide (SiO₂), and glass flake.

4. The emulsion type cosmetic composition according to claim 1, wherein the light interference pigment has an average particle diameter of 2-16 μm.

5. The emulsion type cosmetic composition according to claim 1, which comprises the amphiphilic anisotropic powder in an amount of 0.1-30 wt % based on the total weight of the composition.

6. The emulsion type cosmetic composition according to claim 1, which has a viscosity of 1000-20000 cps.

7. The emulsion type cosmetic composition according to claim 1, which is an oil-in-water type composition.

8. The emulsion type cosmetic composition according to claim 7, wherein the light interference pigment is dispersed in an outer phase.

9. The emulsion type cosmetic composition according to claim 1, wherein the functional group is siloxane.

10. The emulsion type cosmetic composition according to claim 1, wherein the second polymer spheroid and the core of the first polymer spheroid comprise vinyl polymers, and the shell of the first polymer spheroid comprises a copolymer of a vinyl monomer with a functional group-containing monomer.

11. The emulsion type cosmetic composition according to claim 10, wherein the vinyl polymer is a vinyl aromatic polymer.

12. The emulsion type cosmetic composition according to claim 10, wherein the vinyl monomer is a vinyl aromatic monomer.

13. The emulsion type cosmetic composition according to claim 10, wherein the functional group-containing monomer is a siloxane-containing (meth)acrylate.

14. The emulsion type cosmetic composition according to claim 1, wherein the amphiphilic anisotropic powder has a symmetric shape, asymmetric snowman shape or asymmetric reverse snowman shape on the basis of the binding portion where the first polymer spheroid and the second polymer spheroid are bound to each other.

15. The emulsion type cosmetic composition according to claim 1, wherein the amphiphilic anisotropic powder has a particle size of 100-2500 nm.

16. The emulsion type cosmetic composition according to claim 1, wherein the amphiphilic anisotropic powder forms macroemulsion particles having an average particle diameter of 50-300 μm.

17. The emulsion type cosmetic composition according to claim 1, wherein the shell of the first polymer spheroid comprises a hydrophilic functional group introduced additionally thereto.

18. The emulsion type cosmetic composition according to claim 17, wherein the hydrophilic functional group is at least one selected from the group consisting of a carboxylate group, sulfone group, phosphate group, amino group, alkoxy group, ester group, acetate group, polyethylene glycol group and hydroxyl group.

19. The emulsion type cosmetic composition according to claim 1, wherein the shell of the first polymer spheroid comprises a saccharide-containing to functional group additionally introduced thereto.

20. The emulsion type cosmetic composition according to claim 19, wherein the saccharide-containing functional group is derived from at least one selected from the group consisting of N—{N-(3-triethoxysilylpropyl) aminoethyl}gluconamide, N-(3-triethoxysilylpropyl) gluconamide and N—{N-(3-triethoxysilylpropyl)aminoethyl}-oligo-hyaluronamide.
